SEQUENCE LISTING (2 sequences)

SEQ ID NO: 1 - DNA, 21 bp, synthetic construct
  source 1..21: mol_type = other DNA
acgcctgtaatcccagcactt

SEQ ID NO: 2 - DNA, 19 bp, synthetic construct
  source 1..19: mol_type = other DNA
tcgcccaggctggagtgca

(19) United States
(12) Patent Application Publication
(10) Pub. No.: US 2024/0344140 A1
(43) Pub. Date: Oct. 17, 2024

(54) METHODS AND MATERIALS FOR DETECTING COLORECTAL NEOPLASM

(71) Applicant: MAYO FOUNDATION FOR MEDICAL EDUCATION AND RESEARCH, Rochester, MN (US)

(72) Inventors: Hongzhi Zou, Middleton, WI (US); David Ahlquist, Rochester, MN (US); Jonathan J. Harrington, Madison, WI (US)

(21) Appl. No.: 18/594,650

(22) Filed: Mar. 4, 2024

Related U.S. Application Data
(63) Continuation of application No. 16/565,022, filed on Sep. 9, 2019.
Application No. 16/565,022 is a continuation of application No. 14/954,496, filed on Nov. 30, 2015, now Pat. No. 10,435,753.
Application No. 14/954,496 is a continuation of application No. 13/072,047, filed on Mar. 25, 2011, now abandoned.
(60) Provisional application No. 61/318,077, filed on Mar. 26, 2010.

(51) Int. Cl. C12Q 1/6886; G01N 33/574

(52) U.S. Cl. CPC C12Q 1/6886; G01N 33/57419; C12Q 2600/154; C12Q 2600/156; G01N 2333/805; G01N 2800/50; G01N 2800/7028

(57) ABSTRACT

The present invention provides methods and materials related to the detection of colorectal neoplasm-specific markers in or associated with a subject's stool sample. In particular, the present invention provides methods and materials for identifying mammals having a colorectal neoplasm by detecting the presence of exfoliated epithelial markers (e.g., human DNA, tumor associated gene alterations, tumor associated proteins) and blood markers (e.g., hemoglobin, serum proteins) in a stool sample obtained from the mammal.

### CROSS-REFERENCE TO RELATED APPLICATION

The present application is a continuation of U.S. patent application Ser. No. 16/565,022, filed Sep. 9, 2019, which is a continuation of U.S. patent application Ser. No. 14/954,496, filed Nov. 30, 2015, now U.S. Pat. No. 10,435,753, which is a continuation of abandoned U.S. patent application Ser. No. 13/072,047, filed Mar. 25, 2011, which claims priority to U.S. Provisional Patent Application No. 61/318,077, filed Mar. 26, 2010, each of which is incorporated by reference in its entirety.

### SEQUENCE LISTING

The text of the computer readable sequence listing filed herewith, titled “31418-305_SEQUENCE_LISTING”, created Mar. 4, 2024, having a file size of 2,827 bytes, is hereby incorporated by reference in its entirety.

### FIELD OF THE INVENTION

The present invention provides methods and materials related to the detection of colorectal neoplasm-specific markers in or associated with a subject's stool sample. In particular, the present invention provides methods and materials for identifying mammals having a colorectal neoplasm by detecting the presence of exfoliated epithelial markers (e.g., human DNA, tumor associated gene alterations, tumor associated proteins) and blood markers (e.g., hemoglobin, serum proteins) in a stool sample obtained from the mammal.

### BACKGROUND OF THE INVENTION

Colorectal cancer remains a leading cause of death among the types of cancer (see, e.g., Jemal A, et al., CA Cancer J Clin. 2007, 57:43-66; herein incorporated by reference in its entirety). Although screening reduces colorectal cancer mortality (see, e.g., Mandel J S, et al., N Engl J Med. 1993, 328:1365-71; Hardcastle J D, et al., Lancet. 1996, 348:1472-7; Kronborg O, et al., Scand J Gastroenterol. 2004, 39:846-51; Winawer S J, et al., J Natl Cancer Inst. 1993, 85:1311-8; Singh H, et al., JAMA. 2006, 295:2366-73; each herein incorporated by reference in their entireties), observed reductions have been modest (see, e.g., Singh H, et al., JAMA. 2006; 295, 2366-73; Heresbach D, et al., Eur J Gastroenterol Hepatol. 2006, 18:427-33; each herein incorporated by reference in their entireties) and more than one half of adults in the United States have not received screening (see, e.g., Meissner H I, Cancer Epidemiol Biomarkers Prev. 2006, 15:389-94; herein incorporated by reference in its entirety). More accurate, user-friendly, and widely distributable tools to improve screening effectiveness, acceptability, and access are needed.

### SUMMARY

Fecal long DNA originate from either the exfoliation of dysplastic cells or from the luminal hemorrhage or exudation of leukocytes. Previous reports have speculated that long DNA and occult blood in stool resulted from bleeding, and as such, speculated that fecal long DNA testing might be an alternative of fecal occult blood testing (see, e.g., de Kok J B, Clin Chem. 2003; 49:2112-2113; herein incorporated by reference in its entirety). Experiments conducted during preparation of the embodiments for the present invention, however, demonstrated that long DNA was a continuously measurable marker in stool, and occult blood was detectable only intermittently (see, e.g., Osborn N K, et al., Gastroenterology 2005; 128:192-206, Ahlquist D A, Cancer 1989; 63:1826-1830; each herein incorporated by reference in their entireties) thereby indicating that long DNA and occult blood were from different sources and, as such, were complementary markers for CRC detection. Experiments further demonstrated that long DNA and occult blood in stools provide a sensitive approach for the detection of colorectal cancer.

Accordingly, in certain embodiments, the present invention provides methods for detecting the presence of a colorectal neoplasm in a mammal. In some embodiments, the methods involve obtaining a stool sample from a mammal, detecting the presence or absence of one or more exfoliated epithelial markers specific for a colorectal neoplasm in or associated with the stool sample, and detecting the presence or absence of one or more fecal occult blood markers (e.g., specific for a colorectal neoplasm) in the stool sample. In some embodiments, detection of the presence of one or more exfoliated epithelial markers in the stool sample in combination with the presence of one or more fecal occult blood markers in the stool sample is indicative of a colorectal neoplasm in the mammal.

In some embodiments, the mammal is a human. In some embodiments, the colorectal neoplasm is premalignant. In some embodiments, the colorectal neoplasm is malignant.

The methods are not limited to particular exfoliated epithelial markers specific for a colorectal neoplasm. In some embodiments, the one or more colorectal neoplasm-specific nucleic acid markers include, for example, a gene having a point mutation, a gene reflecting microsattelite instability, a gene having aberrant methylation, and/or long DNA.

In some embodiments, a gene having a point mutation includes, but is not limited to, K-ras, APC (see, e.g., U.S. Pat. Nos. 5,352,775, 5,648,212, 5,691,454, 5,783,666, RE36,713, 6,114,124, 6,413,727, and RE38,916; each herein incorporated by reference in their entireties), melanoma antigen gene, p53 (see, e.g., U.S. Pat. Nos. 5,362,623, 5,527,676, 5,955,263, 6,090,566, 6,245,515, 6,677,312, 6,800,617, 7,087,583, 7,267,955; each herein incorporated by reference in their entireties), BRAF, BAT26 and PIK3CA (see, e.g., U.S. patent application Ser. No. 10/591,347; herein incorporated by reference in its entirety). In some embodiments, a gene reflecting microsatellite instability is BAT26. In some embodiments, a gene having aberrant methylation includes, but is not limited to, bmp-3, bmp-4, SFRP2, vimentin (see, e.g., U.S. Pat. No. 7,485,402; herein incorporated by reference in its entirety), septin9, ALX4, EYA4, TFPI2, NDRG4, HLTF (see, e.g., U.S. Pat. No. 7,432,050; herein incorporated by reference in its entirety), and FOXE1. In some embodiments, the long DNA is, for example, greater than 250 base pairs in length, greater than 300 base pairs in length, greater than 400 base pairs in length, greater than 500 base pairs in length, and/or greater than 1000 base pairs in length.

The methods are not limited to particular fecal occult blood markers specific for a colorectal neoplasm. In some embodiments, fecal occult blood markers specific for a colorectal neoplasm include, but are not limited to, hemoglobin, alpha-defensin, calprotectin, al-antitrypsin, albumin, MCM2, transferrin, lactoferrin, and lysozyme.

In some embodiments, the exfoliated epithelial marker specific for colorectal neoplasm is long DNA, and the fecal occult blood marker specific for colorectal neoplasm is hemoglobin.

In some embodiments wherein a colorectal neoplasm is detected, additional techniques are performed to characterise the colorectal neoplasm (e.g., to characterize the colorectal neoplasm as malignant or premalignant) (e.g., to characterize the colorectal neoplasm within a particular stage of colorectal cancer).

In certain embodiments, the present invention provides kits for detecting the presence of a colorectal neoplasm in a mammal. In some embodiments, such kits include reagents useful, sufficient, or necessary for detecting and/or characterizing one or more exfoliated epithelial markers specific for a colorectal neoplasm, and reagents useful, sufficient, or necessary for detecting and/or characterizing one or more fecal occult blood markers specific for a colorectal neoplasm. In some embodiments, the kits contain the reagents necessary to perform real-time Alu PCR. In some embodiments, the kits contain the reagents necessary to perform the heme porphyrin test HemoQuant. In some embodiments, the kits contain the ingredients and reagents necessary to obtain and store a stool sample from a subject.

In certain embodiments, the present invention provides methods for monitoring the treatment of colorectal cancer. For example, in some embodiments, the methods may be performed immediately before, during and/or after a treatment to monitor treatment success. In some embodiments, the methods are performed at intervals on disease free patients to insure or monitor treatment success.

In certain embodiments, the present invention provides methods for obtaining a subject's risk profile for developing colorectal cancer. In some embodiments, such methods involve obtaining a stool sample from a subject (e.g., a human at risk for developing colorectal cancer; a human undergoing a routine physical examination), detecting the presence or absence of one or more exfoliated epithelial markers specific for a colorectal neoplasm in or associated with the stool sample, detecting the presence or absence of one or more fecal occult blood markers (e.g., specific for a colorectal neoplasm) in or associated with the stool sample, and generating a risk profile for developing colorectal cancer based upon the detected presence or absence of the exfoliated epithelial markers and fecal occult blood markers. For example, in some embodiments, a generated risk profile will change depending upon specific exfoliated epithelial markers and fecal occult blood markers detected as present or absent. The present invention is not limited to a particular manner of generating the risk profile. In some embodiments, a processor (e.g., computer) is used to generate such a risk profile. In some embodiments, the processor uses an algorithm (e.g., software) specific for interpreting the presence and absence of specific exfoliated epithelial markers and fecal occult blood markers as determined with the methods of the present invention. In some embodiments, the presence and absence of specific exfoliated epithelial markers and fecal occult blood markers as determined with the methods of the present invention are inputted into such an algorithm, and the risk profile is reported based upon a comparison of such input with established norms (e.g., established norm for pre-cancerous condition, established norm for various risk levels for developing colorectal cancer, established norm for subjects diagnosed with various stages of colorectal cancer). In some embodiments, the risk profile indicates a subject's risk for developing colorectal cancer or a subject's risk for re-developing colorectal cancer. In some embodiments, the risk profile indicates a subject to be, for example, at a very low, a low, a moderate, a high, and a very high chance of developing or re-developing colorectal cancer. In some embodiments, the risk profile indicates risk based on a population average at a desired level of specificity (e.g., 90%). In some embodiments, a health care provider (e.g., an oncologist) will use such a risk profile in determining a course of treatment or intervention (e.g., colonoscopy, wait and see, referral to an oncologist, referral to a surgeon, etc.).

### BRIEF DESCRIPTION OF THE DRAWINGS

FIG. 1A-B shows the levels of long DNA and occult blood in stools from patients with CRCs or advanced adenomas and from normal individuals displayed in log scale. Each circle represents one stool sample.

FIG. 2 shows correlation of long DNA with occult blood levels in stool samples displayed in a scatter plot graph. A black trend is drawn to show that fecal long DNA is not correlated with occult blood levels (R²=0.0001). Because zero cannot be plotted in log scale, long DNA level plus 1 and occult blood level plus 0.1 are displayed here.

FIG. 3A-B shows receiver operating curves for stool long DNA and occult blood levels in patients with colorectal cancers or advanced adenomas versus normal controls. For cancers versus normal controls, AUC values were 0.82, 0.78, and 0.90 for fecal long DNA, occult blood, and combination testing, respectively; for advanced adenomas versus normal controls, AUC values were 0.72, 0.50, and 0.72 for fecal DNA, occult blood, and combination testing, respectively. A, B, and C represent receiver operating curves for fecal long DNA, occult blood, and combination testing, respectively.

### DEFINITIONS

To facilitate an understanding of the present invention, a number of terms and phrases are defined below

As used herein, the term “colorectal cancer” is meant to include the well-accepted medical definition that defines colorectal cancer as a medical condition characterized by cancer of cells of the intestinal tract below the small intestine (e.g., the large intestine (colon), including the cecum, ascending colon, transverse colon, descending colon, and sigmoid colon, and rectum). Additionally, as used herein, the term “colorectal cancer” is meant to further include medical conditions which are characterized by cancer of cells of the duodenum and small intestine (jejunum and ileum).

As used herein, the term “metastasis” is meant to refer to the process in which cancer cells originating in one organ or part of the body relocate to another part of the body and continue to replicate. Metastasized cells subsequently form tumors which may further metastasize. Metastasis thus refers to the spread of cancer from the part of the body where it originally occurs to other parts of the body. As used herein, the term “metastasized colorectal cancer cells” is meant to refer to colorectal cancer cells which have metastasized; colorectal cancer cells localized in a part of the body other than the duodenum, small intestine (jejunum and ileum), large intestine (colon), including the cecum, ascending colon, transverse colon, descending colon, and sigmoid colon, and rectum.

As used herein, “an individual is suspected of being susceptible to metastasized colorectal cancer” is meant to refer to an individual who is at an above-average risk of developing metastasized colorectal cancer. Examples of individuals at a particular risk of developing metastasized colorectal cancer are those whose family medical history indicates above average incidence of colorectal cancer among family members and/or those who have already developed colorectal cancer and have been effectively treated who therefore face a risk of relapse and recurrence. Other factors which may contribute to an above-average risk of developing metastasized colorectal cancer which would thereby lead to the classification of an individual as being suspected of being susceptible to metastasized colorectal cancer may be based upon an individual's specific genetic, medical and/or behavioral background and characteristics.

The term “neoplasm” as used herein refers to any new and abnormal growth of tissue. Thus, a neoplasm can be a premalignant neoplasm or a malignant neoplasm. The term “neoplasm-specific marker” refers to any biological material that can be used to indicate the presence of a neoplasm. Examples of biological materials include, without limitation, nucleic acids, polypeptides, carbohydrates, fatty acids, cellular components (e.g., cell membranes and mitochondria), and whole cells. The term “colorectal neoplasm-specific marker” refers to any biological material that can be used to indicate the presence of a colorectal neoplasm (e.g., a premalignant colorectal neoplasm; a malignant colorectal neoplasm). Examples of colorectal neoplasm-specific markers include, but are not limited to, exfoliated epithelial markes (e.g., bmp-3, bmp-4, SFRP2, vimentin, septin9, ALX4, EYA4, TFPI2, NDRG4, FOXE1, long DNA, BAT-26, K-ras, APC, melanoma antigen gene, p53, BRAF, and PIK3CA) and fecal occult blood markers (e.g., hemoglobin, alpha-defensin, calprotectin, al-antitrypsin, albumin, MCM2, transferrin, lactoferrin, and lysozyme).

As used herein, the term “adenoma” refers to a benign tumor of glandular origin. Although these growths are benign, over time they may progress to become malignant. As used herein the term “colorectal adenoma” refers to a benign colorectal tumor in which the cells form recognizable glandular structures or in which the cells are clearly derived from glandular epithelium.

As used herein, the term “amplicon” refers to a nucleic acid generated using primer pairs. The amplicon is typically single-stranded DNA (e.g., the result of asymmetric amplification), however, it may be RNA or dsDNA.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), Hot-start PCR (see, e.g., U.S. Pat. Nos. 5,773,258 and 5,338,671; each herein incorporated by reference in their entireties), intersequence-specfic PCR, inverse PCR (see, e.g., Triglia, et al. (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93 (13) 9821-9826; herein incorporated by reference in its entirety), miniprimer PCR, multiplex ligation-dependent probe amplification (see, e.g., Schouten, et al., (2002) Nucleic Acids Research 30 (12): e57; herein incorporated by reference in its entirety), multiplex PCR (see, e.g., Chamberlain, et al., (1988) Nucleic Acids Research 16 (23) 11141-11156; Ballabio, et al., (1990) Human Genetics 84 (6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16 (15) 7351-7367; herein incorporated by reference in its entirety), real time PCR (see, e.g., Higuchi, etl al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19 (14) 4008; Roux, K. (1994) Biotechniques 16 (5) 812-814; Hecker, et al., (1996) Biotechniques 20 (3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method. In certain embodiments, the primer is a capture primer.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP).

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68:90-99; the phosphodiester method of Brown et al. (1979) Meth Enzymol. 68:109-151; the diethylphosphoramidite method of Beaucage et al. (1981) Tetrahedron Lett. 22:1859-1862; the triester method of Matteucci et al. (1981) J Am Chem Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

### DETAILED DESCRIPTION OF THE INVENTION

An effective way to detect colorectal cancer (CRC) at early stages is population screening. Colonoscopy and fecal occult blood testing (FOBT) are commonly used tools for CRC screening, but the adherent rates of both approaches are low due to the invasiveness and expense of colonoscopy and the low sensitivity of FOBT (see, e.g., Osborn N K, et al., Gastroenterology 2005; 128:192-206; Levin B, et al., CA Cancer J Clin 2008; 58:130-160; each herein incorporated by reference in its entirety). For example, fecal long DNA quantified with real-time Alu PCR is a simple approach for CRC detection, but its sensitivity is less optimal when assayed alone (see, e.g., Zou H, et al., Cancer Epidemiol Biomarkers Prev 2006; 15:1115-1119; herein incorporated by reference in its entirety).

Fecal long DNA originate from either the exfoliation of dysplastic cells or from the luminal hemorrhage or exudation of leukocytes. Previous reports have speculated that long DNA and occult blood in stool resulted from bleeding, and as such, speculated that fecal long DNA testing might be an alternative of fecal occult blood testing (see, e.g., de Kok J B, Clin Chem. 2003; 49:2112-2113; herein incorporated by reference in its entirety). Experiments conducted during preparation of the embodiments for the present invention, however, demonstrated that long DNA was a continuously measurable marker in stool, and occult blood was detectable only intermittently (see, e.g., Osborn N K, et al., Gastroenterology 2005; 128:192-206, Ahlquist D A, Cancer 1989; 63:1826-1830; each herein incorporated by reference in their entireties) thereby indicating that long DNA and occult blood were from different sources and, as such, were complementary markers for CRC detection. Experiments further demonstrated that long DNA and occult blood in stools provide a sensitive approach for the detection of colorectal cancer.

Ideally, approaches for screening colorectal cancer should be accurate, user friendly, and inexpensive. Both fecal long DNA and occult blood testings are simple non-invasive approaches, but their sensitivities for CRC detection are less optimal when assayed alone (see, e.g., Zou H, et al., Cancer Epidemiol Biomarkers Prev 2006; 15:1115-1119; Mandel J S, et al., N Engl J Med 1993; 328:1365-1371; Kronborg O, et al., Lancet 1996; 348:1467-1471; Hardcastle J D, et al., Lancet 1996; 348:1472-1477; Ahlquist D A, et al., JAMA 1993; 269:1262-1267; Imperiale T F, et al., N Engl J Med 2004; 351:2704-2714; Ahlquist D A, Ann Intern Med 2008; 149:441-450; Ahlquist D A, Gastroenterology 2000; 119:1219-1227; each herein incorporated by reference in their entireties). As experiments conducted during preparation of the embodiments for the present invention demonstrated that stool based long DNA and fecal occult blood are complementary markers originating from different sources, the present invention, in some embodiments, provides a quantitative assay combining exfoliated epithelial markers (e.g., long DNA) and fecal occult blood markers (e.g., hemoglobin) yielding an inexpensive approach for sensitive detection of CRC.

Accordingly, the present invention provides methods and materials related to the detection of colorectal cancer-specific markers in a subject's stool sample. In particular, the present invention provides methods and materials for identifying mammals having a colorectal cancer by detecting the presence of exfoliated epithelial markers (e.g., human long DNA, tumor associated gene alterations, tumor associated proteins) and fecal occult blood markers (e.g., hemoglobin, serum proteins) in a stool sample obtained from the mammal.

While the present invention exemplifies several markers specific for detecting colorectal cancer, any marker that is correlated with the presence or absence of colorectal cancer may be used. A marker, as used herein, includes, for example, any proteinaceous molecule (or corresponding gene) whose production or lack of production is characteristic of a colorectal cancer cell. Depending on the particular set of markers employed in a given analysis, the statistical analysis will vary. For example, where a particular combination of markers is highly specific for colorectal cancer, the statistical significance of a positive result will be high. It may be, however, that such specificity is achieved at the cost of sensitivity (e.g., a negative result may occur even in the presence of colorectal cancer). By the same token, a different combination may be very sensitive (e.g., few false negatives, but has a lower specificity).

Particular combinations of markers may be used that show optimal function with different ethnic groups or sex, different geographic distributions, different stages of disease, different degrees of specificity or different degrees of sensitivity. Particular combinations may also be developed which are particularly sensitive to the effect of therapeutic regimens on disease progression. Subjects may be monitored after a therapy and/or course of action to determine the effectiveness of that specific therapy and/or course of action.

As noted, experiments conducted during the course of developing embodiments for the present invention determined an enhanced method and system for detecting colorectal cancer through detecting and assessing both the presence or absence of exfoliated epithelial markers (e.g., human DNA, tumor associated gene alterations, tumor associated proteins) and fecal occult blood markers (e.g., hemoglobin, serum proteins) in a stool sample obtained from the mammal.

The present invention is not limited to detecting particular exfoliated epithelial markers specific for detecting colorectal cancer. In some embodiments, the exfoliated epithelial markers specific for detecting colorectal cancer include nucleic acid. Examples of colorectal cancer-specific nucleic acid markers include, without limitation, nucleic acid having a point mutation, nucleic acid that reflects microsatellite instability, nucleic acid having aberrant methylation, and long DNA.

Nucleic acid having a point mutation can encode a polypeptide or regulate the expression of a polypeptide (e.g., promoters, enhancers, and silencers). Examples of nucleic acid that can contain a point mutation indicative of a colorectal cancer include, without limitation, the genes for K-ras, APC (adenomatous polyposis coli), melanoma antigen gene, p53, BRAF, BAT26, and PIK3CA.

Nucleic acid that reflects microsatellite instability can be used to indicate the presence of colorectal cancer. Briefly, nucleic acid that reflects microsatellite instability can be identified as described elsewhere (see, e.g., Samowitz et al., Am. J. Path., 154:1637-1641 (1999); Hoang et al., Cancer Res., 57:300-303 (1997); each herein incorporated by reference in its entirety). An example of nucleic acid that can reflect microsatellite instability indicative of a colorectal neoplasm includes, without limitation, the gene for BAT-26.

Nucleic acid having aberrant methylation can be used to indicate the presence of colorectal cancer (see, e.g., Muller, et al., Lancet 2004 363:1283-1285; herein incorporated by reference in its entirety). Examples of nucleic acid having aberrant methylation indicative of colorectal cancer include, but are not limited to, bmp-3, bmp-4, SFRP2, vimentin, septin9, ALX4, EYA4, TFPI2, NDRG4, and FOXE1.

The presence of long DNA in a stool sample is indicative of colorectal cancer. Long DNA is a marker for non-apoptotic cells. Typically, cells shed from normal mucosa are apoptotic, while those shed from colorectal neoplasms are non-apoptotic. As described herein, long DNA can be used as a colorectal cancer-specific marker for subjects having colorectal cancer. One hallmark of apoptosis is the autodigestion or cleavage of DNA into “short” fragments of about 180 base-pairs. The detection of “long” DNA (e.g., DNA greater than about 200 base-pairs) in a stool sample can indicate the presence of non-apoptotic cells of neoplastic lineage derived from a colorectal neoplasm. The term “long DNA” as used herein refers to DNA greater than about 200 base-pairs (e.g., greater than about 250, 300, 350, 400, 500, 600, 700, 800, 900, 1000, 1250, 1500, 1750, 2000, or 2500 base-pairs).

The present invention is not limited to a particular method for detecting and/or quanitifying long DNA within a subject's stool sample. In some embodiments, real-time Alu PCR is used for detecting and/or quanitifying long DNA within a subject's stool sample. Real-time Alu PCR is a sensitive method for detecting non-apoptotic human DNA in stool as it targets abundant Alu repeats in human genome (see, e.g., Zou H, et al. Cancer Epidemiol Biomarkers Prev 2006, 15:1115-1119; herein incorporated by reference in its entirety). Alu sequences embody the largest family of middle repetitive DNA sequences in human genome (see, e.g., Kariya Y, et al., Gene 1987, 53:1-10; herein incorporated by reference in its entirety). An estimated half million Alu copies are present per haploid human genome (see, e.g., Kariya Y, et al., Gene 1987, 53:1-10; herein incorporated by reference in its entirety). Accordingly, as Alu sequences are so abundantly distributed throughout the genome and specific to the genomes primates, real-time Alu PCR amplifies DNA sequences longer than 200 bp within these 300-bp repeats (see, e.g., Kariya Y, et al., Gene 1987, 53:1-10; herein incorporated by reference in its entirety) thereby providing a genome-wide approach to quantify human long DNA in stool (see, e.g., Zou H, et al. Cancer Epidemiol Biomarkers Prev 2006, 15:1115-1119; herein incorporated by reference in its entirety).

The present invention is not limited to detecting particular fecal occult blood markers specific for colorectal cancer. In some embodiments, the fecal occult blood markers specific for detecting colorectal neoplasm include, without limitation, hemoglobin, alpha-defensin, calprotectin, al-antitrypsin, albumin, MCM2, transferrin, lactoferrin, and lysozyme.

Any fecal occult blood testing method can be used to detect colorectal cancer-specific fecal occult blood markers. For example, antibodies specific for a polypeptide marker can be used in an immunoassay (e.g., ELISA) to detect the presence or absence of the polypeptide in a stool sample that is indicative of the presence of colorectal cancer. To accurately evaluate bleeding in the digestive tract, it is important that fecal occult blood tests target analytes that are stable during the enteric transit. Available data indicate that both guaiac and immunochemical FOBTs are insensitive for the detection of bleeding from proximal colon (see, e.g., Imperiale, et al., N Engl J Med 2004, 351:2704-2714; Ahlquist, et al., Ann Intern Med 2008, 149:441-450; Harewood, et al., Mayo Clin Proc 2002, 77:23-28; Allison, et al., J Natl Cancer Inst 2007, 99:1462-1470; each herein incorporated by reference in their entireties). In contrast, the heme porphyrin test HemoQuant is sensitive for both proximal and distal sources of occult bleeding (see, e.g., Harewood, et al., Mayo Clin Proc 2002, 77:23-28; Ahlquist, et al., N Engl J Med 1985, 312:1422-1428; Harewood, et al., Dig Dis. 2000, 18 (2): 75-82; each herein incorporated by reference in their entireties). Accordingly, in some embodiments, the heme porphyrin test HemoQuant is used for detecting and/or quantifying the colorectal cancer-specific fecal occult blood marker hemoglobin.

The present invention is not limited to a particular combination of exfoliated epithelial markers and fecal occult blood markers in the detection of colorectal cancer in a subject. In some embodiments, any one or more exfoliated epithelial markers are used (e.g., bmp-3, bmp-4, SFRP2, vimentin, septin9, ALX4, EYA4, TFPI2, NDRG4, FOXE1, long DNA, BAT-26, K-ras, APC, melanoma antigen gene, p53, BRAF, and PIK3CA). In some embodiments, any one or more fecal occult blood markers are used (e.g., hemoglobin, alpha-defensin, calprotectin, al-antitrypsin, albumin, MCM2, transferrin, lactoferrin, and lysozyme).

It is noted that a single stool sample can be analyzed for one colorectal neoplasm-specific marker or for multiple colorectal neoplasm-specific markers. For example, a stool sample can be analyzed using assays that detect a panel of different colorectal neoplasm-specific markers. In addition, multiple stool samples can be collected for a single mammal and analyzed as described herein. Indeed, U.S. Pat. Nos. 5,670,325, 5,741,650, 5,928,870, 5,952,178, and 6,020,137, each herein incorporated by reference in their entireties, for example, describe various methods that can be used to prepare and analyze stool samples. In some embodiments, a stool sample is split into first and second portions, where the first portion undergoes analysis for exfoliated epithelial markers and the second portion undergoes analysis for fecal occult blood markers. In some embodiments, the stool sample undergoes one or more preprocessing steps before being split into portions.

The present invention is not limited to a particular manner of detecting nucleic acid markers corresponding to colorectal neoplasm from a stool sample. In some embodiments, nucleic acid is amplified. Generally, nucleic acid used as template for amplification is isolated from cells contained in the biological sample according to standard methodologies (see, e.g., Sambrook, J., et al., Fritsch, E. F., Maniatis, T. (ed.). MOLECULAR CLONING. Cold Spring Harbor Lab. Press, Cold Spring Harbor, N.Y. (1989); herein incorporated by reference in its entirety). The nucleic acid may be genomic DNA or fractionated or whole cell RNA. Where RNA is used, it may be desired to convert the RNA to a complementary cDNA. In a preferred embodiment, the RNA is whole cell RNA and is used directly as the template for amplification. Pairs of primers that selectively hybridize to genes corresponding to specific markers are contacted with the isolated nucleic acid under conditions that permit selective hybridization. Once hybridized, the nucleic acid primer complex is contacted with one or more enzymes that facilitate template-dependent nucleic acid synthesis. Multiple rounds of amplification, also referred to as “cycles,” are conducted until a sufficient amount of amplification product is produced. Next, the amplification product is detected. In some applications, the detection may be performed by visual means. Alternatively, the detection may involve indirect identification of the product via chemiluminescence, radioactive scintigraphy of incorporated radio label or fluorescent label or even via a system using electrical or thermal impulse signals. Generally, the foregoing process is conducted at least twice on a given sample using at least two different primer pairs specific for two different specific markers. Following detection, in some embodiments, the results seen in a given subject are compared with a statistically significant reference group of subjects diagnosed as not having colorectal cancer.

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred.

In most cases, it will be preferable to synthesize desired oligonucleotides. Suitable primers can be synthesized using commercial synthesizers using methods well known to those of ordinary skill in the art. Where double-stranded primers are desired, synthesis of complementary primers is performed separately and the primers mixed under conditions permitting their hybridization.

Selection of primers is based on a variety of different factors, depending on the method of amplification and the specific marker involved. For example, the choice of primer will determine the specificity of the amplification reaction. The primer needs to be sufficiently long to specifically hybridize to the marker nucleic acid and allow synthesis of amplification products in the presence of the polymerization agent and under appropriate temperature conditions. Shorter primer molecules generally require cooler temperatures to form sufficiently stable hybrid complexes with the marker nucleic acid and may be more susceptible to non-specific hybridization and amplification.

Primer sequences do not need to correspond exactly to the specific marker sequence. Non-complementary nucleotide fragments may be attached to the 5′ end of the primer with the remainder of the primer sequence being complementary to the template. Alternatively, non-complementary bases can be interspersed into the primer, provided that the primer sequence has sufficient complementarily, in particular at the 3′ end, with the template for annealing to occur and allow synthesis of a complementary DNA strand.

In some embodiments, primers may be designed to hybridize to specific regions of the marker nucleic acid sequence. For example, GC rich regions are favored as they form stronger hybridization complexes than AT rich regions. In another example, primers are designed, solely, to hybridize to a pair of exon sequences, with at least one intron in between. This allows for the activity of a marker gene to be detected as opposed to its presence by minimizing background amplification of the genomic sequences and readily distinguishes the target amplification by size.

Primers also may be designed to amplify a particular segment of marker nucleic acid that encodes restriction sites. A restriction site in the final amplification product would enable digestion at that particular site by the relevant restriction enzyme to produce two products of a specific size. Any restriction enzyme may be utilized in this aspect. This added refinement to the amplification process may be necessary when amplifying a marker nucleic acid sequence with close sequence similarity to other nucleic acids. Alternatively, it may be used as an added confirmation of the specificity of the amplification product.

A number of template dependent processes are available to amplify the marker sequences present in a given template sample. One of the best known amplification methods is the polymerase chain reaction (PCR) (see, e.g., U.S. Pat. Nos. 4,683,195, 4,683,202, 4,800,159, and Innis et al., PCR Protocols, Academic Press, Inc., San Diego, Calif. (1990); each incorporated herein by reference in their entireties). Briefly, in PCR, two primer sequences are prepared which are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated. In some embodiments, a reverse transcriptase PCR amplification procedure is performed in order to quantify the amount of mRNA amplified. Methods of reverse transcribing RNA into cDNA are well known (see, e.g., Sambrook, J., et al., Fritsch, E. F., Maniatis, T. (ed.). MOLECULAR CLONING. Cold Spring Harbor Lab. Press, Cold Spring Harbor, N.Y. (1989); herein incorporated by reference in its entirety). Alternatively, methods for reverse transcription utilize thermostable DNA polymerases (see, e.g., WO 90/07641; herein incorporated by reference in its entirety).

The present invention is not limited to a particular PCR technique. Examples of PCR include, but are not limited to, standard PCR, allele-specific PCR, assembly PCR, asymmetric PCR, digital PCR, helicase-dependent amplification, Hot-start PCR, interseqeunce-specfic PCR, inverse PCR, ligation-mediated PCR, methylation-specific PCR, miniprimer PCR, multiplex ligation-dependent probe amplification, nested PCR, overlap-extension PCR, real-time PCR, reverse transcription PCR, solid phase PCR, thermal asymmetric interlaced PCR, and Touchdown PCR.

Another method for amplification is the ligase chain reaction (“LCR”) (see, e.g., U.S. Pat. Nos. 4,883,750 and 5,494,810; herein incorporated by reference in its entirety). In LCR, two complementary probe pairs are prepared, and in the presence of the marker sequence, each pair will bind to opposite complementary strands of the marker such that they abut. In the presence of a ligase, the two probe pairs will link to form a single unit. By temperature cycling, as in PCR, bound ligated units dissociate from the marker and then serve as “target sequences” for ligation of excess probe pairs.

Following amplification, it may be desirable to separate the amplification product from the template and the excess primer for the purpose of determining whether specific amplification occurred. In some embodiments, amplification products are separated by agarose, agarose-acrylamide or polyacrylamide gel electrophoresis using standard methods (see, e.g., Sambrook, J., et al., Fritsch, E. F., Maniatis, T. (ed.). MOLECULAR CLONING. Cold Spring Harbor Lab. Press, Cold Spring Harbor, N.Y. (1989); herein incorporated by reference in its entirety).

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (see, e.g., Freifelder, D. Phpysical Biochemistry Applications to Biochemistry and Molecular Biology. 2nd ed. Wm. Freeman & Co., New York, N.Y. 1982; incorporated herein by reference in its entirety).

Amplification products must be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In some embodiments, visualization is achieved indirectly. For example, following separation of amplification products, a nucleic acid probe is brought into contact with the amplified marker sequence. The probe preferably is conjugated to a chromophore but may be radiolabeled. In another embodiment, the probe is conjugated to a binding partner, such as an antibody or biotin, where the other member of the binding pair carries a detectable moiety.

In some embodiments, detection is by Southern blotting and hybridization with a labeled probe. The techniques involved in Southern blotting are well known to those of skill in the art and can be found in many standard books on molecular protocols (see, e.g., Sambrook, J., et al., Fritsch, E. F., Maniatis, T. (ed.). MOLECULAR CLONING. Cold Spring Harbor Lab. Press, Cold Spring Harbor, N.Y. (1989); herein incorporated by reference in its entirety). Briefly, amplification products are separated by gel electrophoresis. The gel is then contacted with a membrane, such as nitrocellulose, permitting transfer of the nucleic acid and non-covalent binding. Subsequently, the membrane is incubated with a chromophore conjugated probe that is capable of hybridizing with a target amplification product. Detection is by exposure of the membrane to x-ray film or ion-emitting detection devices.

In some embodiments, all the basic essential materials and reagents required for detecting colorectal cancer through detecting both the presence or absence of exfoliated epithelial markers (e.g., human DNA, tumor associated gene alterations, tumor associated proteins) and fecal occult blood markers (e.g., hemoglobin, serum proteins) in a stool sample obtained from the mammal are assembled together in a kit. Such kits generally comprise, for example, reagents useful, sufficient, or necessary for detecting and/or characterizing one or more exfoliated epithelial markers specific for a colorectal neoplasm (e.g., bmp-3, bmp-4, SFRP2, vimentin, septin9, ALX4, EYA4, TFPI2, NDRG4, FOXE1, long DNA, BAT-26, K-ras, APC, melanoma antigen gene, p53, BRAF, and PIK3CA), and reagents useful, sufficient, or necessary for detecting and/or characterizing one or more fecal occult blood markers specific for a colorectal neoplasm (e.g., hemoglobin, alpha-defensin, calprotectin, al-antitrypsin, albumin, MCM2, transferrin, lactoferrin, and lysozyme). In some embodiments, the kits contain enzymes suitable for amplifying nucleic acids including various polymerases, deoxynucleotides and buffers to provide the necessary reaction mixture for amplification. In some embodiments, the kits contain reagents necessary to perform Real-time Alu PCR. In some embodiments, the kits contain reagents necessary to perform the heme porphyrin test HemoQuant. In some embodiments, the kits of the present invention include a means for containing the reagents in close confinement for commercial sale such as, e.g., injection or blow-molded plastic containers into which the desired reagent are retained. Other containers suitable for conducting certain steps of the disclosed methods also may be provided.

In some embodiments, the methods disclosed herein are useful in monitoring the treatment of colorectal cancer. For example, in some embodiments, the methods may be performed immediately before, during and/or after a treatment to monitor treatment success. In some embodiments, the methods are performed at intervals on disease free patients to insure treatment success.

The present invention also provides a variety of computer-related embodiments. Specifically, in some embodiments the invention provides computer programming for analyzing and comparing a pattern of colorectal neoplasm-specific marker (e.g., exfoliated epithelial markers and fecal occult blood markers) detection results in a stool sample obtained from a subject to, for example, a library of such marker patterns known to be indicative of the presence or absence of a colorectal cancer, or a particular stage or colorectal cancer.

In some embodiments, the present invention provides computer programming for analyzing and comparing a first and a second pattern of colorectal neoplasm-specific marker (e.g., exfoliated epithelial markers and fecal occult blood markers) detection results from a stool sample taken at at least two different time points. In some embodiments, the first pattern may be indicative of a pre-cancerous condition and/or low risk condition for colorectal cancer and/or progression from a pre-cancerous condition to a cancerous condition. In such embodiments, the comparing provides for monitoring of the progression of the condition from the first time point to the second time point.

In yet another embodiment, the invention provides computer programming for analyzing and comparing a pattern of colorectal neoplasm-specific marker (e.g., exfoliated epithelial markers and fecal occult blood markers) detection results from a stool sample to a library of colorectal neoplasm-specific marker patterns known to be indicative of the presence or absence of a colorectal cancer, wherein the comparing provides, for example, a differential diagnosis between a benign colorectal neoplasm, and an aggressively malignant colorectal neoplasm (e.g., the marker pattern provides for staging and/or grading of the cancerous condition).

The methods and systems described herein can be implemented in numerous ways. In one embodiment, the methods involve use of a communications infrastructure, for example the internet. Several embodiments of the invention are discussed below. It is also to be understood that the present invention may be implemented in various forms of hardware, software, firmware, processors, or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site (e.g., at a service provider's facility).

For example, during or after data input by the user, portions of the data processing can be performed in the user-side computing environment. For example, the user-side computing environment can be programmed to provide for defined test codes to denote platform, carrier/diagnostic test, or both; processing of data using defined flags, and/or generation of flag configurations, where the responses are transmitted as processed or partially processed responses to the reviewer's computing environment in the form of test code and flag configurations for subsequent execution of one or more algorithms to provide a results and/or generate a report in the reviewer's computing environment.

The application program for executing the algorithms described herein may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device and a printing device.

As a computer system, the system generally includes a processor unit. The processor unit operates to receive information, which generally includes test data (e.g., specific gene products assayed), and test result data (e.g., the pattern of colorectal neoplasm-specific marker (e.g., exfoliated epithelial markers and fecal occult blood markers) detection results from a stool sample). This information received can be stored at least temporarily in a database, and data analyzed in comparison to a library of marker patterns known to be indicative of the presence or absence of a pre-cancerous condition, or known to be indicative of a stage and/or grade of colorectal cancer.

Part or all of the input and output data can also be sent electronically; certain output data (e.g., reports) can be sent electronically or telephonically (e.g., by facsimile, e.g., using devices such as fax back). Exemplary output receiving devices can include a display element, a printer, a facsimile device and the like. Electronic forms of transmission and/or display can include email, interactive television, and the like. In some embodiments, all or a portion of the input data and/or all or a portion of the output data (e.g., usually at least the library of the pattern of colorectal neoplasm-specific marker (e.g., exfoliated epithelial markers and fecal occult blood markers) detection results known to be indicative of the presence or absence of a pre-cancerous condition) are maintained on a server for access, e.g., confidential access. The results may be accessed or sent to professionals as desired.

A system for use in the methods described herein generally includes at least one computer processor (e.g., where the method is carried out in its entirety at a single site) or at least two networked computer processors (e.g., where detected marker data for a stool sample obtained from a subject is to be input by a user (e.g., a technician or someone performing the activity assays)) and transmitted to a remote site to a second computer processor for analysis (e.g., where the pattern of colorectal neoplasm-specific marker (e.g., exfoliated epithelial markers and fecal occult blood markers) detection results is compared to a library of patterns known to be indicative of the presence or absence of a pre-cancerous condition), where the first and second computer processors are connected by a network, e.g., via an intranet or internet). The system can also include a user component(s) for input; and a reviewer component(s) for review of data, and generation of reports, including detection of a pre-cancerous condition, staging and/or grading of a colorectal neoplasm, or monitoring the progression of a pre-cancerous condition or a colorectal neoplasm. Additional components of the system can include a server component(s); and a database(s) for storing data (e.g., as in a database of report elements, e.g., a library of marker patterns known to be indicative of the presence or absence of a pre-cancerous condition and/or known to be indicative of a grade and/or a stage of a colorectal neoplasm, or a relational database (RDB) which can include data input by the user and data output. The computer processors can be processors that are typically found in personal desktop computers (e.g., IBM, Dell, Macintosh), portable computers, mainframes, minicomputers, or other computing devices.

The input components can be complete, stand-alone personal computers offering a full range of power and features to run applications. The user component usually operates under any desired operating system and includes a communication element (e.g., a modem or other hardware for connecting to a network), one or more input devices (e.g., a keyboard, mouse, keypad, or other device used to transfer information or commands), a storage element (e.g., a hard drive or other computer-readable, computer-writable storage medium), and a display element (e.g., a monitor, television, LCD, LED, or other display device that conveys information to the user). The user enters input commands into the computer processor through an input device. Generally, the user interface is a graphical user interface (GUI) written for web browser applications.

The server component(s) can be a personal computer, a minicomputer, or a mainframe and offers data management, information sharing between clients, network administration and security. The application and any databases used can be on the same or different servers.

Other computing arrangements for the user and server(s), including processing on a single machine such as a mainframe, a collection of machines, or other suitable configuration are contemplated. In general, the user and server machines work together to accomplish the processing of the present invention.

Where used, the database(s) is usually connected to the database server component and can be any device which will hold data. For example, the database can be any magnetic or optical storing device for a computer (e.g., CDROM, internal hard drive, tape drive). The database can be located remote to the server component (with access via a network, modem, etc.) or locally to the server component.

Where used in the system and methods, the database can be a relational database that is organized and accessed according to relationships between data items. The relational database is generally composed of a plurality of tables (entities). The rows of a table represent records (collections of information about separate items) and the columns represent fields (particular attributes of a record). In its simplest conception, the relational database is a collection of data entries that “relate” to each other through at least one common field.

Additional workstations equipped with computers and printers may be used at point of service to enter data and, in some embodiments, generate appropriate reports, if desired. The computer(s) can have a shortcut (e.g., on the desktop) to launch the application to facilitate initiation of data entry, transmission, analysis, report receipt, etc. as desired.

In certain embodiments, the present invention provides methods for obtaining a subject's risk profile for developing colorectal cancer. In some embodiments, such methods involve obtaining a stool sample from a subject (e.g., a human at risk for developing colorectal cancer; a human undergoing a routine physical examination), detecting the presence or absence of one or more exfoliated epithelial markers specific for a colorectal neoplasm in or associated with the stool sample, detecting the presence or absence of one or more fecal occult blood markers (e.g., specific for a colorectal neoplasm) in or associated with the stool sample, and generating a risk profile for developing colorectal cancer based upon the detected presence or absence of the exfoliated epithelial markers and fecal occult blood markers. For example, in some embodiments, a generated risk profile will change depending upon specific exfoliated epithelial markers and fecal occult blood markers detected as present or absent. The present invention is not limited to a particular manner of generating the risk profile. In some embodiments, a processor (e.g., computer) is used to generate such a risk profile. In some embodiments, the processor uses an algorithm (e.g., software) specific for interpreting the presence and absence of specific exfoliated epithelial markers and fecal occult blood markers as determined with the methods of the present invention. In some embodiments, the presence and absence of specific exfoliated epithelial markers and fecal occult blood markers as determined with the methods of the present invention are inputted into such an algorithm, and the risk profile is reported based upon a comparison of such input with established norms (e.g., established norm for pre-cancerous condition, established norm for various risk levels for developing colorectal cancer, established norm for subjects diagnosed with various stages of colorectal cancer). In some embodiments, the risk profile indicates a subject's risk for developing colorectal cancer or a subject's risk for re-developing colorectal cancer. In some embodiments, the risk profile indicates a subject to be, for example, a very low, a low, a moderate, a high, and a very high chance of developing or re-developing colorectal cancer. In some embodiments, the risk profile indicates risk based on a population average at a desire level of specificity (e.g., 90%). In some embodiments, a health care provider (e.g., an oncologist) will use such a risk profile in determining a course of treatment or intervention (e.g., colonoscopy, wait and see, referral to an oncologist, referral to a surgeon, etc.).

### EXAMPLES

The invention now being generally described, will be more readily understood by reference to the following example, which is included merely for purposes of illustration of certain aspects and embodiments of the present invention, and are not intended to limit the invention.

### Example 1

### Subjects

A total of two hundred and one subjects, including 74 patients with CRC, 27 with advanced adenoma (≥1 cm), and 100 colonoscopically normal individuals, were all recruited. Their demographic and clinical characteristics are described in Table 1.

TABLE 1
Demographic and clinical characteristics of subjects

| | Colorectal | Advanced | |
|---|---|---|---|
| | cancer | adenoma | Normal control |
| Number | 74 | 27 | 100 |
| Median Age (Range) | 61 (40-87) yrs | 67 (50-82) yrs | 59 (28-81) yrs |
| Sex (Male/Female) | 52/22 | 15/12 | 37/63 |
| Site (Right/Left) | 29/45 | 17/10 | |
| Stage (I/II/III/IV) | 13/16/27/18 | | |
| Grade (1/2/3/4) or | 0/4/55/5 | 20/5 | |
| Dysplasia (Low/High) | | | |

### Stool Collection

Stools were collected more than 2 weeks following any colorectal diagnostic procedure or cathartic preparation and prior to either endoscopic or surgical neoplasm resection. Patients collected whole stools in a preservative buffer (0.5 mol/L Tris, 10 mmol/L NaCl, 150 mmol/L EDTA, pH 9.0) as described (see, e.g., Olson J, et al., Diagn Mol Pathol 2005; 14:183-191; herein incorporated by reference in its entirety), and mailed to a laboratory within 48 hours. Once a stool arrived in the laboratory, it was weighed and homogenized. One aliquot equivalent to 10 g stool was used for stool DNA extraction, and the rest was stored at −80° C. in aliquots.

Long DNA Quantification with Real-Time Alu PCR

Crude stool DNA was extracted with isopropanol, precipitated with ethanol, and eluted in 7.5 ml 1×TE buffer. Human DNA in crude stool DNA was quantified using a real-time Alu PCR method (see, e.g., Zou H, et al., Cancer Epidemiol Biomarkers Prev 2006; 15:1115-1119; herein incorporated by reference in its entirety). Primers specific for the human Alu sequences, sense: 5′-ACG CCT GTA ATC CCA GCA CTT-3′ (SEQ ID NO: 1); and antisense: 5′-TCG CCC AGG CTG GAG TGC A-3′ (SEQ ID NO: 2) were used to amplify sequences about 245 bp inside Alu repeats (see, e.g., Zou H, et al., Cancer Epidemiol Biomarkers Prev 2006; 15:1115-1119; Zijlstra A, et al., Cancer Res 2002; 62:7083-7092; each herein incorporated by reference in their entireties). Crude stool DNA was diluted 1 to 100 with nuclease-free water for PCR amplification. One μL water-diluted stool DNA was amplified in a total volume of 25 μL containing 1×iQ™ SYBR® Green Supermix (BioRad), 200 nM each primer under the following conditions: 95° C. for 3 minutes, followed by 30 cycles of 95° C. for 30 seconds, 60° C. for 30 seconds, and 72° C. for 40 seconds in a real-time iCycler™ (BioRad). Standard curve was created for each plate by amplifying 10-fold serially diluted human genomic DNA samples (Novagen, Madison, WI). Melting curve was made after each PCR reaction to confirm that only one product was amplified for all samples. Amplification was carried out in 96-well plates. Each plate consisted of stool DNA samples and multiple positive and negative controls. Each assay was performed in duplicate.

Fecal Occult Blood Quantification with HemoQuant

Fecal occult blood was quantified with HemoQuant test (see, e.g., Harewood G C, et al., Mayo Clin Proc 2002; 77:23-28; Ahlquist D A, et al., JAMA 1993; 269:1262-1267; Ahlquist D A, et al., N Engl J Med 1985; 312:1422-1428; Ahlquist D A, et al., Ann Intern Med 1984; 101:297-302; Schwartz S, et al., Clin Chem 1983; 29:2061-2067; Schwartz S, et al., Gastroenterology 1985; 89:19-26; each herein incorporated by reference in their entireties). Buffer (0.5 mol/L Tris, 10 mmol/L NaCl, 150 mmol/L EDTA, pH 9.0) (see, e.g., Olson J, et al., Diagn Mol Pathol 2005; 14:183-191; herein incorporated by reference in its entirety) preserved frozen stool slurry equivalent to 8 mg stool was used to perform HemoQuant test with an automated system in a clinical lab. Non-fluorescing heme in stool sample was converted to fluorescing porphyrins by removal of iron with 2 mL hot (60° C.) oxalic acid and 0.2 mL ferrous sulfate. The reaction mixture was then extracted with 3 mL ethyl acetate:isobutyl alcohol (11.1:1, v/v) followed by washing with 3 mL alkalinized aqueous solution of potassium acetate, and further extracted with 2.5 mL acetic acid/phosphoric acid. The extracted porphyrins, mostly protoporphyrin, are quantified based on fluorescence emission intensity at 652 nm using 405 nm as the excitation wavelength in an Infinite® 200 microplate reader (Tecan, Männedorf, Switzerland). Each plate consisted stool samples, standards, and negative and positive controls. Standards were made with known amounts of hemoglobin from human blood.

### Statistical Analysis

Logistic procedure was used to calculate the correlation of stool long DNA and hemoglobin levels. Wilcoxon Rank Sum test was used to compare the long DNA or hemoglobin levels between each of the three different stool groups, and evaluate the association of marker levels with tumor location, gender, Dukes stage, and differentiation grade. The correlation of marker levels with tumor size and patient age was calculated with Logistic procedure. Chi-Square and Fisher exact tests were used to evaluate the association of detection rates of marker panel with clinical characteristics. Combination of long DNA and hemoglobin levels was calculated with a logistic model. Receiver Operating Curve (ROC) was constructed to compare long DNA and hemoglobin levels in cancers or adenomas versus normal subjects, and area under the curve (AUC) value was also calculated for each curve. Sensitivities were calculated at 90% specificity for single markers and their combination. Statistical analysis was conducted with SAS software (SAS Institute, Cary, NC).

### Results

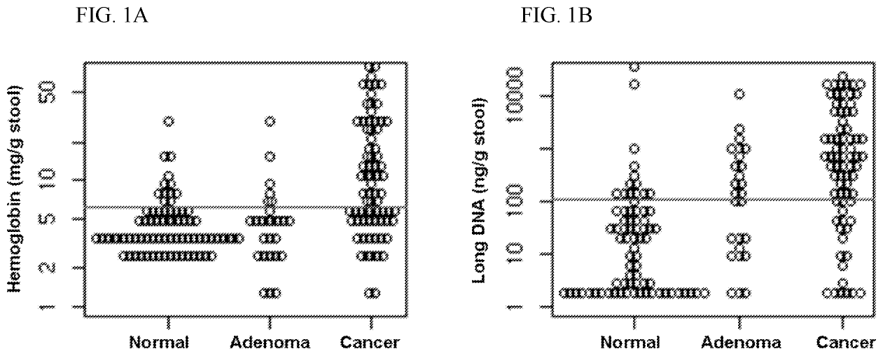

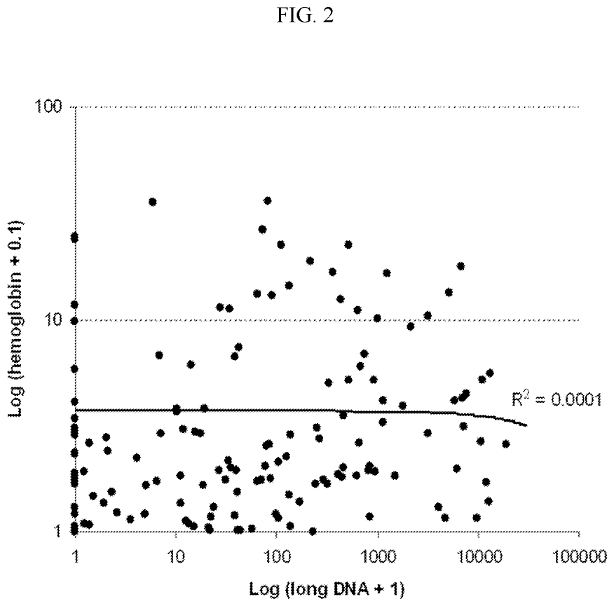

The median human DNA levels were 421 (range; 0-19140), 86 (0-6260), and 11 (0-30000) ng per g stools from patients with CRCs or adenomas, or from normal controls, respectively (p=0.0001, CRC vs normal; p=0.0005, adenoma vs. normal; p=0.007, CRC vs adenoma; FIG. 1A). The median hemoglobin levels were 3.4 (0.1-36.1), 1.5 (0.1-10.9), and 1.1 (0.3-11.0) mg per g stools from patients with CRCs or adenomas, or from normal controls, respectively (p=0.0001, CRC vs. normal or adenoma; p=1.0, adenoma vs normal; FIG. 1B). Fecal long DNA and occult blood levels were not correlated (R²=0.0001; FIG. 2).

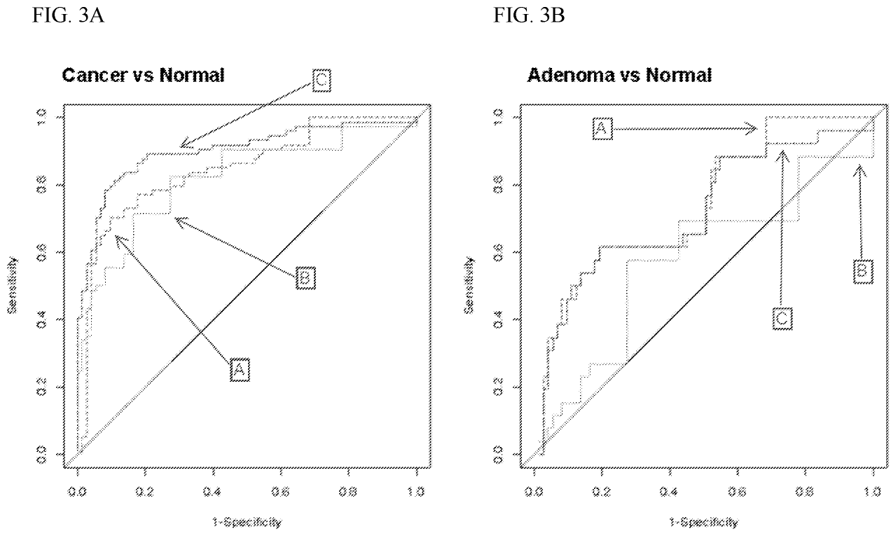

At 90% specificities, long DNA testing detected 70% of colorectal cancers and 46% of adenomas while occult blood testing detected 50% and 12% (FIGS. 1 & 3). Combining these tests detected 80% colorectal cancers and 46% of adenomas at 90% specificity (FIG. 3). For detection of colorectal cancer, AUC values were 0.82, 0.78, and 0.90 for fecal long DNA, occult blood, and combination testing, respectively (p=0.02, long DNA vs combination; p=0.0001, occult blood vs. combination; FIG. 3). For detection of advanced adenoma, AUC values were 0.72, 0.50, and 0.72 for fecal DNA, occult blood, and combination testing, respectively (p=0.8, long DNA vs combination; p=0.03, occult blood vs. combination; FIG. 3).

The median (range) fecal long DNA level was 181 (0-7120) ng/g stool with stage 1-2 cancers and 910 (0-19140) ng/g stool with stage 3-4 cancers, p=0.001; and 112 (0-4780) ng/g stool with proximal cancers and 1006 (0-19140) ng/g stool with distal cancers, p=0.0001. The median fecal occult blood level was 7.0 mg Hb/g (0.2-26.4 mg/g) with proximal cancers and 2.5 mg/g (0.1-36.1 mg/g) with distal ones, p=0.04. The median fecal long DNA and occult blood levels in stools from patients with CRCs and advanced adenomas were not associated with other clinical characteristics. Median size was 4.0 cm (1.0-15.0) for neoplasms detected by the combined tests and 3.7 cm (1.0-7.0) for neoplasms missed, p=0.02. Neoplasm detection rates by combined tests were affected by neither tumor site nor stage.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1.-18. (canceled)

19. A method of characterizing a stool sample from a human subject, comprising:
a) homogenizing the stool sample with a buffer to form a homogenized sample;
b) extracting DNA from the homogenized sample to form extracted DNA;
c) contacting the extracted DNA with one or more reagents that amplify DNA indicative of human long DNA, if human long DNA is present in the extracted DNA; and
d) measuring an amount of DNA amplified from the extracted DNA with the one or more reagents, wherein the amount of DNA amplified from the extracted DNA with the one or more reagents is indicative of an amount of human long DNA in the stool sample.

20. The method of claim 19, wherein the human long DNA comprises DNA greater than 250 base pairs in length.

21. The method of claim 19, wherein the human long DNA comprises DNA greater than 300 base pairs in length.

22. The method of claim 19, wherein the human long DNA comprises DNA greater than 400 base pairs in length.

23. The method of claim 19, wherein the human long DNA comprises DNA greater than 500 base pairs in length.

24. The method of claim 19, wherein the human long DNA comprises DNA greater than 1000 base pairs in length.

25. The method of claim 19, wherein the one or more reagents that amplify DNA indicative of human long DNA amplify Alu sequences in human DNA.

26. The method of claim 19, further comprising contacting the extracted DNA with one or more reagents that amplify human DNA indicative of one or more exfoliated epithelial markers indicative of a human gene having a point mutation, a human gene having aberrant methylation, and/or a human gene reflecting microsatellite instability.

27. The method of claim 26, wherein the one or more exfoliated epithelial markers are selected from the group consisting of bmp-3, bmp-4, SFRP2, vimentin, septin9, ALX4, EYA4, TFPI2, NDRG4, FOXE1, BAT-26, K-ras, APC, melanoma antigen gene, p53, BRAF, and PIK3CA DNAs.

28. The method of claim 26, wherein the human gene having a point mutation is selected from the group consisting of K-ras, APC, melanoma antigen gene, p53, BRAF, BAT26 and PIK3CA.

29. The method of claim 26, wherein the human gene reflecting microsatellite instability is BAT26.

30. The method of claim 26, wherein the human gene having aberrant methylation is selected from the group consisting of bmp-3, bmp-4, SFRP2, vimentin, septin9, ALX4, EYA4, TFPI2, NDRG4, and FOXE1.

31. The method of claim 19, further comprising detecting in the stool sample the presence or absence of one or more fecal occult blood markers.

32. The method of claim 31, wherein the one or more fecal occult blood markers are selected from the group consisting of hemoglobin, alpha-defensin, calprotectin, al-antitrypsin, albumin, MCM2, transferrin, lactoferrin, and lysozyme.

33. The method of claim 31, wherein the one or more fecal occult blood markers are detected through a heme porphyrin test.

34. The method of claim 31, wherein the one or more fecal occult blood markers are detected through a guaiac test.

35. The method of claim 31, wherein the one or more fecal occult blood markers are detected through an immunochemical test.
